(19) United States
(12) Patent Application Publication
(10) Pub. No.: US 2020/0129241 A1
(43) Pub. Date: Apr. 30, 2020

(54) SURGICAL MARKERS FOR ROBOTIC SURGERY WITH REDUCED BONE PENETRATION

(71) Applicant: Think Surgical, Inc., Fremont, CA (US)

(72) Inventors: Micah Forstein, Fremont, CA (US); Steve Whiseant, Fremont, CA (US); Pauline Biasbas, Fremont, CA (US)

(21) Appl. No.: 16/669,176

(22) Filed: Oct. 30, 2019

Related U.S. Application Data
(60) Provisional application No. 62/752,714, filed on Oct. 30, 2018.

(51) Int. Cl. A61B 34/20; A61B 34/32; A61B 90/00

(52) U.S. Cl. CPC A61B 34/20; A61B 34/32; A61B 90/39; A61B 2034/2065; A61B 2090/3983; A61B 2034/207; A61B 2090/3916; A61B 2090/395; A61B 2090/3966; A61B 2034/2059

(57) ABSTRACT

A process is provided to recover a bone-to-image, image-to-system, and/or bone-to-system registration, when bone movement occurs during a computer-assisted surgical procedure. The process may be implemented with a computer-assisted surgical system. Examples of surgical systems that can exploit the recovery process illustratively include a 1-6 degree of freedom hand-held surgical system, a navigated surgical system, a serial-chain manipulator system, a parallel robotic system, or a master-slave robotic system.

### RELATED APPLICATIONS

This application claims priority benefit of U.S. Provisional Application Ser. No. 62/752,714 filed 30 Oct. 2018, the contents of which are hereby incorporated by reference.

### TECHNICAL FIELD

The present invention generally relates to computer-assisted surgery, and in particular to a method and system for recovering a registration during a computer-assisted surgical procedure.

### BACKGROUND

Registration of an object (e.g., a rigid bone during a total joint replacement procedure) requires matching the surface of the bone to a pre-existing virtual model, or image data set of the bone. Once the object moves (relative to a fixation system or tracking array), a recovery of the registration is rapidly achieved by locating points on recovery markers that have been placed in known locations on the object prior to the registration. Several methods of registration recovery are known in the art. In one method, recovery of all six degrees of freedom (6-DOF) can be accomplished by locating three known points on two recovery markers as described in U.S. Pat. No. 6,430,434. However, this method requires the removal of a portion of one of the recovery markers to collect a point, which may inadvertently introduce error in the registration recovery process.

Another method is to use three individual recovery markers placed on the bone to define the three points. This adds an additional marker to be placed on the bone. Depending on the surgical access to the operating site, this may be difficult, and may also increase the overall surgical procedure duration.

A third method may use two recovery markers placed on the bone to define the first two recovery points. The third point is collected on the bone in a general area away from the operating site (e.g., the femoral shaft in total hip replacement procedures) using a percutaneous probe. Although this method is accurate, it requires the surgeon to pierce the skin with little knowledge of the underlying anatomy such as critical nerves, arteries and veins.

In certain medical procedures, such as robotic surgery, precision and accuracy are critical. Although robotic surgeries are accurate and repeatable, the surgical times may be increased compared to conventional or manual procedures. Therefore, an efficient registration recovery method is essential in the event the object moves post registration. Movement of the object is observed to occur even with a retentive limb securing device, as the tension on the bone is modified in the course of separating tendons, muscles and otherwise progressing through the procedure.

FIG. 1 depicts an example of a prior art point recovery marker 110 as described in U.S. Pat. No. 6,430,434. The prior art point recovery marker 110 includes a divot 112 for facilitating the collection of a fixed point, and a bone engaging portion 114 (e.g., a spike, a pin) for engaging a bone. Other markers may include a divot positioned on one end of a shaft, where the opposing end of the shaft has threads that screw into the bone. Registration recovery currently requires the use of these markers, which can be intrusive, difficult to place, and might weaken the bone.

Additionally, for many total joint replacement procedures, it may be desirable to monitor the position of the implant overtime. Often, the implant begins to subside or move, which can affect the patient's quality of life and eventually require a revision. Currently, monitoring the position of an implant post-op is difficult to perform accurately without x-rays, bone modeling, and extensive image processing techniques.

Thus, there exists a need for a method to recover a registration of an object, such as a bone or joint after bone movement during a robotic surgical procedure that requires minimally invasive techniques, markings, and hardware while maintaining excellent accuracy. There further exists a need for a method to recover a registration of an object that is quicker than the aforementioned methods while maintaining the needed degree of accuracy. There is an even further need for a method to quickly assess the position of an implant post-operatively, to monitor any movement of the implant overtime.

### SUMMARY

A method is provided for re-registration of a bone-to-image, image-to-system, or bone-to-system registration for computer-assisted surgery. The method includes providing at least one of a bone-to-image, image-to-system, or bone-to-system registration based upon an initial position of the bone relative to a coordinate system of the system; locating a first point, a second point, and a third point fixed relative to the initial bone position prior to any detectable change in bone position from the initial bone position, where the first point, the second point, and third point are defined by a ink tattoo that is stamped or imprinted on the bone or a physical feature made in the bone; relocating the first point, second point, and third point after bone motion may have occurred to determine a locational change in the first point, second point, and third point; and re-registering at least one of the bone-to-image, image-to-system, or bone-to-system based on the locational change to continue a computer-assisted surgery.

A method is provided for re-registration of a bone-to-image, image-to-system, or bone-to-system registration for computer-assisted surgery. The method includes providing at least one of a bone-to-image, image-to-system, or bone-to-system registration based upon an initial position of the bone relative to a coordinate system of the system; locating a first point and a groove fixed relative to the initial bone position prior to any detectable change in bone position from the initial bone position, where the first point and the groove are defined by an ink tattoo that is stamped or imprinted on the bone or a physical feature made in the bone; relocating the first point and at least a portion of the groove after bone motion may have occurred to determine a locational change in the first point and the groove; and re-registering at least one of the bone-to-image, image-to-system, or bone-to-system based on the locational change to continue a computer-assisted surgery.

A system is provided for registration recovery of a bone. The system includes a first point, second point, and third point formed with an ink tattoo that is stamped or imprinted on the bone or a physical feature made in the bone. The system further includes a fiducial marker array adjustably fixed to the bone, and a probe tracked by a tracking system for collecting one or more points on the first point, the second point, and the third point.

A system is provided for registration recovery of a bone. The system includes a first point, a second point, and a third point formed with an ink tattoo that is stamped or imprinted on the bone or a physical feature made in the bone, and a robotic system positioned adjacent to the bone having at least one of a robotic arm and a mechanical digitizer arm, where the robotic arm or digitizer arm are used to collect a point on each of the first point, the second point, and the third point to permit registration recovery of the bone.

### BRIEF DESCRIPTION OF THE DRAWINGS

The present invention is further detailed with respect to the following drawings that are intended to show certain aspects of the present of invention, but should not be construed as limits on the practice of the invention, wherein:

FIG. 1 depicts a prior art point registration recovery marker;

FIG. 2A illustrates the placement of three marker points that are representative of printed markers or a drilled-in/incised markers in a bone for use as recovery markers in accordance with embodiments of the invention;

FIG. 2B depicts a process for collecting a set of points that are representative of printed markers or a drilled-in/incised markers in a bone and calculating a plurality of vectors from those points to detect bone movement in accordance with embodiments of the invention;

FIG. 3A illustrates the placement of a marker point and a groove that are representative of a printed marker and groove or a drilled-in/incised marker and groove in a bone for use as recovery markers in accordance with embodiments of the invention;

FIG. 3B depicts a process for collecting a point and line that are representative of a printed marker and groove or a drilled-in/incised marker and groove in a bone and calculating a plurality of vectors from the point and groove prior to bone motion in accordance with embodiments of the invention;

FIG. 3C depicts a process for collecting a point and line that are representative of a printed marker and groove or a drilled-in/incised marker and groove in a bone and calculating a plurality of vectors from the point and groove post bone motion in accordance with embodiments of the invention;

FIG. 4 depicts a bone having a tracking array and three marker points that are representative of printed markers or a drilled-in/incised markers in a bone for use as recovery markers in accordance with embodiments of the invention; and

FIG. 5 depicts a robotic system for recovering a registration of a bone in accordance with embodiments of the invention.

### DETAILED DESCRIPTION OF THE INVENTION

The present invention has utility as a method to recover a bone-to-image, image-to-system, and/or bone-to-system registration, when bone movement occurs during a computer-assisted surgical procedure. The following description of the preferred embodiments of the invention is not intended to limit the invention to these preferred embodiments, but rather to enable any person skilled in the art to make and use this invention.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

Embodiments of the present invention may be implemented with a computer-assisted surgical system. Examples of surgical systems that can exploit embodiments of the invention illustratively include a 1-6 degree of freedom hand-held surgical system, a navigated surgical system, a serial-chain manipulator system, a parallel robotic system, or a master-slave robotic system, as described in the following patents and patent applications, all of which are incorporated by reference herein in their entirety: U.S. Pat. Nos. 5,086,401, 6,033,415, 7,206,626, 7,803,158 8,876,830, 8,961,536, and 9,707,043; and U.S. Pat. App. Pub. No. 2017/0258532. The computer-assisted surgical system may provide autonomous, semi-autonomous, haptic, or no control (passive), and any combinations thereof.

Also, referenced herein is the use of a mechanical digitizer arm and an optically tracked probe for collecting various points in the operating room. An example of a mechanical digitizer arm for collecting points is described in U.S. Pat. No. 6,430,434, and an example of an optically tracked probe for collecting points is described in U.S. Pat. No. 7,043,961, both of which are hereby incorporated by reference herein in their entirety. For clarity, the “collection of points” refers to the digitizing, measuring, and/or recording of the location of physical points in space into a reference coordinate frame/system, such as the coordinate frame of a robotic system or a tracking system.

In a specific inventive embodiment, an ink tattoo is stamped or imprinted on a bone to be used as a marker for registration recovery and registering the bone post-operation. Embodiments of the ink may be radio opaque for post case registration using X-rays, fluoroscopy, or MRI imaging. The ability to use x-ray imaging to detect the position of the bone eliminates the need for the use of permanent invasive screw in mechanisms such as screws or tacks for bone registration. The use of a stamped or imprinted ink is particularly advantageous as the ink is not interfering with the implant, and can be used for post-operative registration to monitor the implant in the bone overtime. By way of example, such inks illustratively include iron-oxide (brown) and beta-carotene (orange) pigments are encapsulated in polymer beads of polymethyl methacrylate (PMMA), gentian violet, biocompatible inks used with surgical marking pens, and biocompatible inks used for tattoos.

In a specific inventive embodiment, physical features may be made in or on a bone in lieu of physical implanted recovery markers that may cause damage to the bone through the implantation. For example, drilled-in recovery points may be used to replace the currently installed recovery markers. The drilled-in recovery points may be small indentations made to the bone, by a drilling apparatus. The drilled-in recovery points are sized to be as small as the holes in existing recovery markers. It is appreciated that a notch or groove may also be scribed as a physical feature made in the bone. A set of temporary labels may be used to locate the drilled recovery markers in a bone. In some inventive embodiments, the physical features do not penetrate the cortical bone. In some embodiments, the stamped or imprinted ink may likewise be used in lieu of physically implanted recovery markers. If the ink is only used for registration recovery, the ink may be bioresorbable.

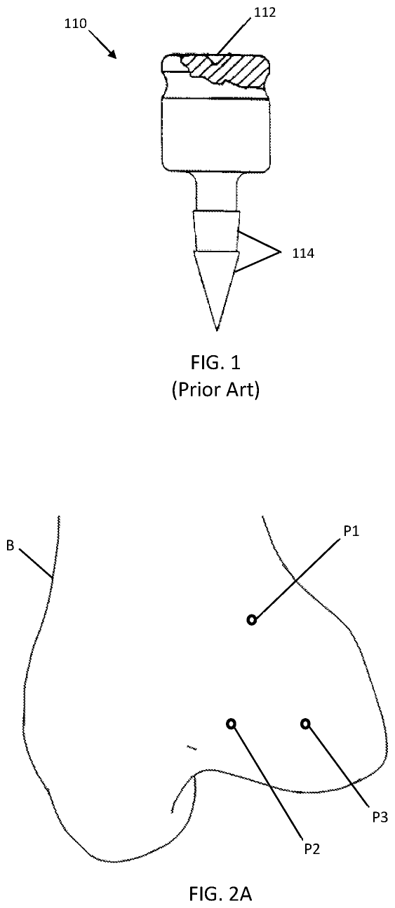

A small drill may be used to make the correct diameter and depth (hard stop) of the one or more drilled-in recovery markers in a subject bone. The depth of the drilled hole is controlled by a hard stop, and the drill or etch tool can only go as far as it is allowed. The drilled-in recovery markers are approximately the same size as the current indentations of prior art recovery markers such as art point recovery marker 110 that includes a divot 112 as shown in FIG. 1. The drilled-in recover markers may then be labeled, to help in locating the drilled holes, due to their small size. The drilled holes may then be used as the recovery points. The shallow depth and small diameter of the drilled locator holes cause less damage to the bone as only a small pin hole is drilled instead of what is currently used for physical markers. In other embodiments, the physical features (e.g., holes, groove) may be made incised on the bone with a pin, tack, nail, or other pointed instrument. The user may incise the hole on the bone by pushing the pin or tack against the bone, thereby removing or compressing that portion of the bone to form the hole. A user may further stain, stamp, or imprint the drilled-in markers/incisions with an ink as described herein.

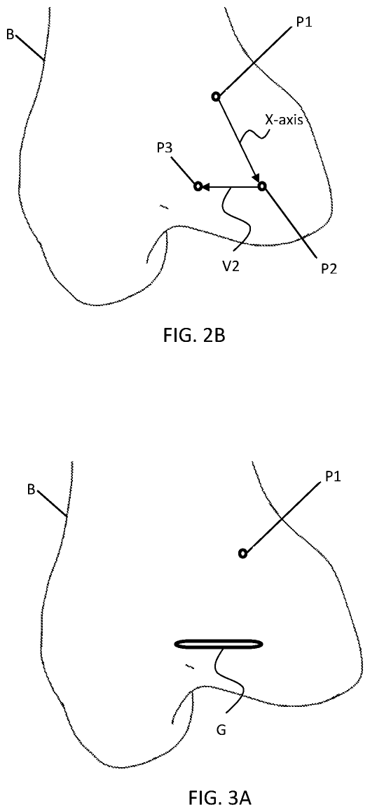

Referring now to the figures, FIG. 2A illustrates a bone B shown with a set of three points P1, P2, and P3 either printed on the surface of the bone, or partially drilled or incised into the bone at a limited depth. Once the three markers are installed, a registration technique known in the art such as point-to-surface as described in U.S. Pat. No. 6,033,415 may be used to register: (i) the bone B to an image of the bone (bone-to-image); (ii) an image of the bone to the surgical system based on the position of the bone B (image-to-system); (iii) the bone B directly to the system (bone-to-system); or a combination of any or all of the aforementioned. In some inventive embodiments, the bone is registered with respect to the coordinates of a tracking system, a computer-assisted surgical system, or both.

With respect to FIG. 2B, a fully-defined coordinate system on the bone is determined to track the bone in the event of bone motion. Conserved points P1, P2, and P3 are non-collinear and thereby define a three-dimensional coordinate system. As points P1, P2, and P3 remain fixed relative to the bone B, motion of any or all of points P1, P2, and P3 will identically track bone motion. The locations of the conserved points P1, P2, and P3 are used to define a coordinate system as follows. After the points P1, P2, and P3 are printed or drilled-in or incised onto the bone, each point is digitized to determine their location in physical space. An x-axis is then defined between points P1 and P2 as follows:

$$Xaxis = \frac{P 2 - P 1}{‖ P 2 - P 1 ‖}$$

Another vector V2 can be defined by P3 and P2:

$$V 2 = \frac{P 3 - P 2}{‖ P 3 - P 2 ‖}$$

The Y-axis is then defined as the unit vector of the cross product of the X-axis and V2, and the Z-axis is defined as the cross product of the X and Y axes.

After bone motion, the three points P1, P2, and P3 are re-digitized. The transformation matrix between the pre-bone-motion coordinate system and the post-bone-motion coordinate system is determined. This transformation matrix defines the movement of the bone and is applied to recover the registration.

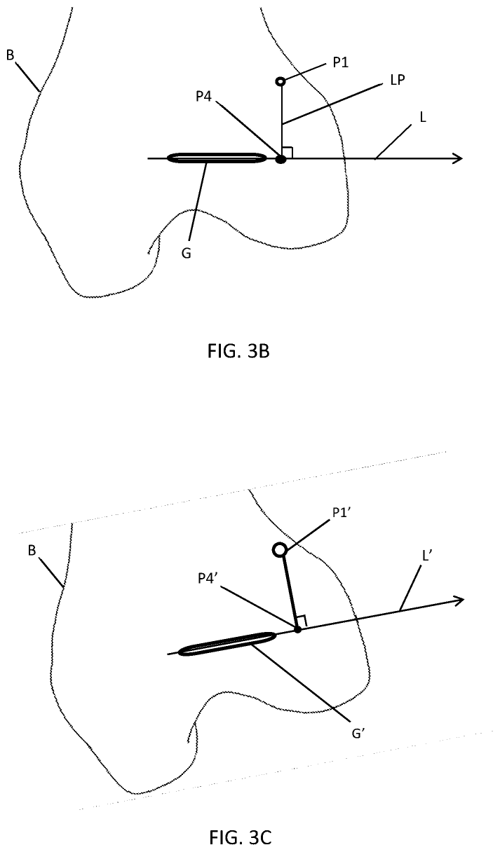

With respect to FIGS. 3A-3C, another embodiment for recovering the registration is shown. FIG. 3A illustrates a bone B shown with a point P1 and groove G either printed on the surface of the bone, or partially drilled or incised into the bone at a limited depth. Once the marker P1 and groove G are installed, a registration technique known in the art such as point-to-surface as described in U.S. Pat. No. 6,033,415 may be used to register: (i) the bone B to an image of the bone (bone-to-image); (ii) an image of the bone to the surgical system based on the position of the bone B (image-to-system); (iii) the bone B directly to the system (bone-to-system); or a combination of any or all of the aforementioned. In some inventive embodiments, the bone is registered with respect to the coordinates of a tracking system, a computer-assisted surgical system, or both.

With reference to FIG. 3B, prior to, or after bone registration, but before bone motion the following procedure is executed:
a. A distal end of a passive digitizer arm, a tracked probe, or a distal end of a robotic surgical system is used to collect fixed point P1 in the printed indicia or in the partially drilled hole in the bone.
b. A distal end of a passive digitizer arm, a tracked probe, or a distal end of a robotic surgical system is used to collet several points (e.g., 10 or more) along the groove G. The user can simply slide the distal end or probe tip along the groove G while the system (e.g., the computer-assisted surgical system, a mechanical digitizer system, or a tracking system) acquires the several points during the sliding action.
c. The system fits a straight line L to the several acquired points and determines a vector in the direction away from the bone surface (arrow L).
d. The system calculates a point P4 on the line L that is the closest to P1 (i.e., the perpendicular distance from point P1 to line L. Point P4 is on a line LP through P1 that is perpendicular to line L.
e. This defines all 6-DOF.

With reference to FIG. 3C, after bone motion the following procedure is executed to recover the registration:
f. Acquire several points on the groove G′ going away from the bone surface as described in step b.
g. The system fits a straight line L′ from the acquired points and determines a vector in the direction away from the bone surface.
h. Collect fixed point P1′ on the bone B.
i. The system calculates the point P4′ on the line L′ closest to point P1′ using the same method as step d.
j. The system determines any translational movement, T, of the bone from point P4 to point P4′ by subtraction T=P4′-P4.
k. The system determines any rotational movement, R, of the bone by finding the unit vector about which a single rotation is done, and the scalar amount of rotation angle, theta.
   i. The unit vector about which a single rotation is done can be calculated by taking the cross product between a unit vector along L and a unit vector along L′.
   ii. The scalar amount of rotation angle, theta, can be calculated by taking the dot product between a unit vector along L and a unit vector along L′.
l. Combine T and R to obtain the transformation matrix M.

The resultant 4×4 matrix M is the matrix for a rigid transformation that completely describes the six degrees of freedom of bone movement. This transformation is used to re-register at least one of the bone-to-image, image-to-system, and/or bone-to-system. In a specific embodiment, the image-data set of the bone is re-registered within a computer-assisted surgical system coordinates or a tracking system coordinates using the transformation matrix M without having to repeat a full registration technique such as point-to-surface registration. Except for theta, all symbols here are math vectors.

The present invention provides a speed advantage over the conventional methods of re-registration methods such as those detailed herein. Conventional methods require the installation of physical markers in the bone and in strategic locations to not interfere with the surgical procedure. The present method permits the markers to be imprinted, drilled-in, or incised anywhere on the bone without worry of interference. Additionally, the imprinting, drilling, or incising of the markers onto the bone is particularly faster than having to drill-in physical markers. In some instances, the speed advantage is two to three times faster than the same procedure performed with conventional re-registration methods. The practical implications of the present invention are that a robotic surgery proceeds at times that are competitive with manual surgery and even faster than manual surgery with the added benefit of more accurate cuts and less collateral bone damage associated with fixturing cutting jigs to the bones. As a result, implant fit is improved and healing time reduced leading to better overall surgical results, all while doing so on at least a similar time frame compared to comparable manual surgery.

Post-Operative Registration

Imprinted ink markers are particularly advantageous for registering the bone post-operation to monitor movement of an implant relative to the bone overtime. After the procedure, an imaging technique (e.g., X-rays, computed-tomography) images the bone and the implant. The markers are visible on the images and are fixed relative to the placement of the bone and the implant. These images may be saved for registration with subsequent images of the bone and implant. For example, after 6 months, 1 year, 5 years, etc. the bone and implant may be re-imaged. The visible markers may be directly registered with the visible marks on the previous images without any extensive image processing currently needed. Once registered, the previous position of the implant relative to the bone can be compared with the current position of the implant relative to the bone. Ideally, the positions of the implants are perfectly aligned. If the positions of the implants are not aligned however, then the implant has moved. Thus, a quick assessment of the implant position can be monitored overtime. The use of the tattoo marker for post-op registration greatly decreases the time to assess implant position compared to conventional techniques. The post-op registration speed using the markers is on the order of minutes compared to hours with conventional techniques.

Optical Tracking and Registration Recovery

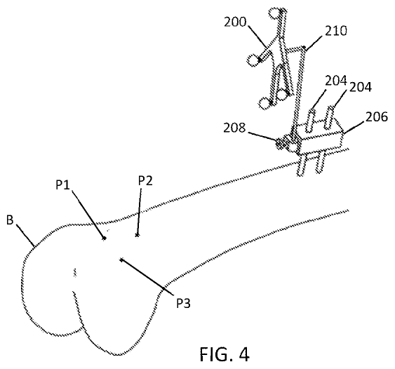

In a specific inventive embodiment, with reference to FIG. 4, a system and method for recovering a registration of a tracked bone is shown. FIG. 4 depicts a fiducial marker array 200 fixed to the bone B to track the bone with an optical tracking system. The fiducial marker array 200 is fixed to the bone using various components prior to registering the bone B. The components may include two bone pins 204 drilled into the bone, a pin clamp 206 for clamping to the pins 204 in a fixed relative orientation to the bone, and an array clamp 208 for clamping a rod 210 attached to the fiducial marker array 200 to the pin clamp 206. The array clamp 208 is adjustable to allow the user to easily orient the fiducial marker array 200 towards a pair of optical receivers of an optical tracking system prior to clamping and securing the POSE of the array 200 relative to the bone B.

It is imperative that the POSE of the marker array 200 remains rigid with respect to the bone B after the bone B is initially registered; otherwise, the tracking is no longer accurate. Conventionally, a method to quickly determine whether the marker array 200 has moved relative to the bone B, involves the use of a checkpoint marker. The checkpoint marker is a physical marker inserted directly on the bone B to ensure the checkpoint marker does not move relative to the bone B. Throughout the course of a procedure, the POSE of the marker array 200 relative to the bone B is checked by digitizing the checkpoint marker 202 and determining if the relative POSE of the marker array 200 with respect to the checkpoint marker 202 has changed. If a change is detected at any two time points during the procedure then the marker array 200 has moved relative to the bone B, and the registration and tracking is no longer accurate. In this case, the surgeon must fully re-register the bone using a time consuming registration technique such as point-to-surface. To avoid having to fully re-register the above methods may be used with the tracking array 200 to quickly re-register the bone B.

Since there is always a chance the marker array 200 may move relative to the bone B, the registration recovery method described herein is well suited for recovering the registration in such a case. The checkpoint marker is replaced with an imprinted, drilled-in, or incised marker P1, P2, or P3. After the marker array 200 is fixed with respect to the bone B, the methods described above is executed to recover the registration in the event the marker array 200 moves relative to the bone B. The markers P1, P2, and P3 may likewise be used to determine if the fiducial marker array 200 has moved relative to the bone. Therefore, the markers P1, P2, and P3 are particularly advantageous to quickly recover the registration and further determine if the array 200 has moved relative to the bone B. It should be appreciated, that although a fiducial marker array 200 is illustratively shown, the same method may be applied to other tracking systems such as a mechanical tracking system having a probe fixed directly to the bone B.

Robotic System

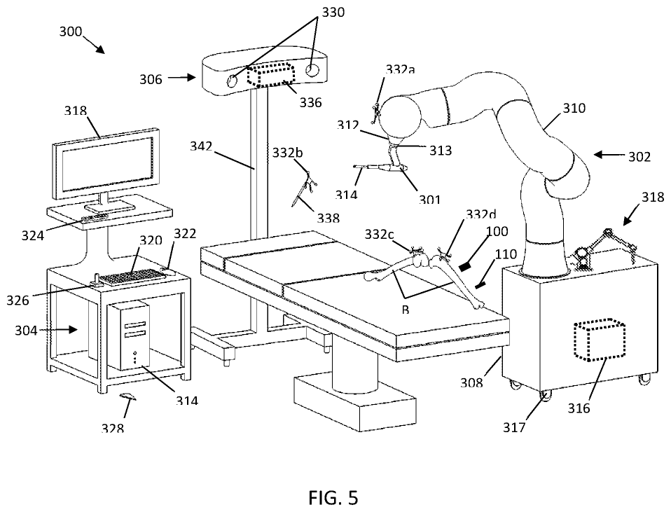

With reference to FIG. 5, an embodiment of a robotic surgical system is shown generally at 300 capable of implementing embodiments of the inventive method for registration recovery of a bone. The surgical system 300 generally includes a surgical robot 302, a computing system 304, and may include at least one of a mechanical digitizer arm 318 and/or an optical tracking system 306.

The surgical robot 302 may include a movable base 308, a manipulator arm 310 connected to the base 308, an end-effector flange 312 located at a distal end of the manipulator arm 310, and an end-effector assembly 301 for holding and/or operating a tool 314 removably attached to the flange 312 by way of an end-effector mount 313. The base 308 may include an actuator to adjust the height of the robotic arm 310. The base may further include a set of wheels 317 to maneuver the base 308, which may be fixed into position using a braking mechanism such as a hydraulic brake. The manipulator arm 310 includes various joints and links to manipulate the tool 314 in various degrees of freedom. If a mechanical digitizer 318 or optical tracking system 306 is not present, the tool 314 may be fitted with a probe tip to collect points on the recovery markers (P1, P2, P3) directly. The joints are illustratively prismatic, revolute, or a combination thereof.

The computing system 304 generally includes a planning computer 314; a device computer 316; a tracking computer 336 if a tracking system 306 is present; and peripheral devices. The planning computer 314, device computer 316, and tracking computer 336, may be separate entities, single units, or combinations thereof depending on the surgical system. The peripheral devices allow a user to interface with the surgical system components and may include: one or more user-interfaces, such as a display or monitor 318; and user-input mechanisms, such as a keyboard 320, mouse 322, pendent 324, joystick 326, foot pedal 328, or the monitor 318 in some inventive embodiments have touchscreen capabilities.

The planning computer 314 contains hardware (e.g., processors, controllers, and memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan includes operational data for modifying a volume of tissue that is defined relative to the anatomy, such as a set of points in a cut-file to autonomously modify the volume of bone, a set of virtual boundaries defined to haptically constrain a tool within the defined boundaries to modify the bone, a set of planes or drill holes to drill pins in the bone, or a graphically navigated set of instructions for modifying the tissue. The data generated from the planning computer 314 may be transferred to the device computer 316 and/or tracking computer 336 through a wired or wirelessly connection in the operating room (OR); or transferred via a non-transient data storage medium (e.g., a compact disc (CD), a portable universal serial bus (USB) drive) if the planning computer 314 is located outside the OR.

The device computer 316 in some inventive embodiments is housed in the moveable base 308 and contains hardware, software, data and utilities that are preferably dedicated to the operation of the surgical device 302. This may include surgical device control, robotic manipulator control, the processing of kinematic and inverse kinematic data, the execution of registration algorithms, the execution of calibration routines, the execution of surgical plan data, coordinate transformation processing, providing workflow instructions to a user, and utilizing position and orientation (POSE) data from the tracking system 306.

The optional tracking system 306 of the surgical system 300 includes two or more optical receivers 330 to detect the position of fiducial markers (e.g., retroreflective spheres, active light emitting diodes (LEDs)) uniquely arranged on rigid bodies. The fiducial markers arranged on a rigid body are collectively referred to as a fiducial marker array 332, where each fiducial marker array 332 has a unique arrangement of fiducial markers, or a unique transmitting wavelength/frequency if the markers are active LEDs. An example of an optical tracking system is described in U.S. Pat. No. 6,061,644. The tracking system 306 may be built into a surgical light, located on a boom, a stand 342, or built into the walls or ceilings of the OR. The tracking system computer 336 may include tracking hardware, software, data and utilities to determine the POSE of objects (e.g., bones B, surgical device 304) in a local or global coordinate frame. The POSE of the objects is collectively referred to herein as POSE data, where this POSE data may be communicated to the device computer 316 through a wired or wireless connection. Alternatively, the device computer 316 may determine the POSE data using the position of the fiducial markers detected from the optical receivers 330 directly.

The POSE data is determined using the position data detected from the optical receivers 330 and operations/processes such as image processing, image filtering, triangulation algorithms, geometric relationship processing, registration algorithms, calibration algorithms, and coordinate transformation processing. For example, the POSE of a digitizer probe 338 with an attached probe fiducial marker array 332b may be calibrated such that the probe tip is continuously known as described in U.S. Pat. No. 7,043,961. The POSE of the tool tip or tool axis of the tool 314 may be known with respect to a device fiducial marker array 332a using a calibration method as described in U.S. Pat. Pub. No. 2018/0014888. The device fiducial marker 332a is depicted on the manipulator arm 310 but may also be positioned on the base 308 or the end-effector assembly 301. Registration algorithms may be executed to determine the POSE and coordinate transforms between a bone B, a fiducial marker array 332c or 332d, and a surgical plan, using the registration methods described in U.S. Pat. Nos. 6,033,415, and 8,287,522.

Upon assembly of the device tracking array 332a to the surgical robot 302 prior to surgery, the POSE's of the coordinate systems, 332a and the end effector tool 314, are fixed relative to each other and stored in memory to accurately track the end effector tool 314 during the surgery (see for example U.S. Pat. No. 9,480,534) relative to the bone anatomy (e.g., bones B). The POSE data may be used by the computing system 304 during the procedure to update the bone and surgical plan coordinate transforms so the surgical robot 302 can accurately execute the surgical plan in the event any bone motion occurs. However, if there is unintentional movement between the fiducial marker arrays (332c, 332d) and the bone B after initially registering the bone B, then the bone needs to be re-registered to re-establish the coordinate systems between the fiducial marker arrays (332c, 332d) and the bone B. It should be appreciated that in certain embodiments, other tracking systems may be incorporated with the surgical system 300 such as an electromagnetic field tracking system or a 6-DOF mechanical tracking system. An example of a 6-DOF mechanical tracking system is described in U.S. Pat. No. 6,322,567. In a particular inventive embodiment, the surgical system 300 does not include a tracking system 306, but instead employs a bone fixation and monitoring system that fixes the bone directly to the surgical robot 302 in the robotic coordinate frame and monitors bone movement as described in U.S. Pat. No. 5,086,401.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method for re-registration of a bone-to-image, image-to-system, or bone-to-system registration for computer-assisted surgery, said method comprising:
providing at least one of a bone-to-image, image-to-system, or bone-to-system registration based upon an initial position of the bone relative to a coordinate system of the system;
locating a first point, a second point, and a third point fixed relative to the initial bone position or a first point and a groove fixed relative to the initial bone position, prior to any detectable change in bone position from the initial bone position, and wherein the first point, the second point, and third point; or the first point and the groove are defined by a ink tattoo that is stamped or imprinted on the bone or a physical feature made in the bone;
relocating the first point, second point, and third point; or the first point and the groove after bone motion may have occurred to determine a locational change in the first point, second point, and third point; or the first point and the groove; and
re-registering at least one of the bone-to-image, image-to-system, or bone-to-system based on the locational change to continue a computer-assisted surgery.

2. The method of claim 1, wherein at least one of the first point, the second point, or the third point are made after the image data set has been created.

3. The method of claim 1, wherein locating the first point, the second point, and the third point is accomplished by contacting a distal end of a passive mechanical digitizing arm against the first point, the second point, and the third point, respectively.

4. The method of claim 1, wherein locating the first point, the second point, and the third point is accomplished by contacting a distal end of a surgical robotic arm against the first point, the second point, and the third point, respectively.

5. The method of claim 1, wherein locating the first point, second point, and third point is accomplished by contacting a distal end of a tracked probe against the first point, the second point, and the third point, respectively.

6. The method of claim 1 wherein the system is at least one a computer-assisted surgical system or a tracking system.

7. The method of claim 1 wherein the physical feature is at least one of a drilled hole, notch, or groove.

8. The method of claim 1 wherein the ink is radio opaque.

9. The method of claim 1 wherein said re-registering is two to three times faster than a like procedure performed with conventional re-registration methods.

10. A system for registration recovery of a bone comprising:
a first point, second point, and third point formed with an ink tattoo that is stamped or imprinted on the bone or a physical feature made in the bone;
a fiducial marker array adjustably fixed to the bone; and
a probe tracked by a tracking system for collecting one or more points on the first point, the second point, and the third point.

11. The system of claim 10 further comprising a computer for calculating the re-registration of the bone according to the method of claim 1.

12. The system of claim 10 wherein the physical feature is at least one of a drilled hole, incised hole, notch, or groove.

13. The system of claim 10 wherein the ink is radio opaque.

14. A system for registration recovery of a bone comprising:
a first point, a second point, and a third point formed with an ink tattoo that is stamped or imprinted on the bone or a physical feature made in the bone;
a robotic system positioned adjacent to the bone having at least one of a robotic arm and a mechanical digitizer arm wherein the robotic arm or digitizer arm are used to collect a point on each of the first point, the second point, and the third point to permit registration recovery of the bone.

15. The system of claim 14 wherein the physical feature is at least one of a drilled hole, incised hole, notch, or groove.

16. The system of claim 14 wherein the ink is radio opaque.

17. The system of claim 14 wherein the robotic system comprises a robotic arm and a mechanical digitizer arm wherein the mechanical digitizer arm is used to collect the point on the point recovery marker and the series of points on the groove recovery marker.

18. The system of claim 14 wherein the bone is fixed relative to a robotic coordinate frame of the robotic system.

19. The system of claim 14 further comprising a computer to calculate the re-registration of the bone according to the method of claim 1.
